(19) United States
(12) Patent Application Publication
(10) Pub. No.: US 2021/0200065 A1
(43) Pub. Date: Jul. 1, 2021

(54) LENS DEVICE WITH SHADING STRUCTURE

(71) Applicant: CAL-COMP BIG DATA, INC., New Taipei City (TW)

(72) Inventors: Ming-Hua HUNG, NEW TAIPEI CITY (TW); Chu-Chiang WENG, NEW TAIPEI CITY (TW)

(21) Appl. No.: 16/825,198

(22) Filed: Mar. 20, 2020

(30) Foreign Application Priority Data
Dec. 27, 2019 (CN) 201911388004.0

(51) Int. Cl. G03B 17/02; G02B 7/04; H04N 5/225

(52) U.S. Cl. CPC G03B 17/02; H04N 5/2254; G02B 7/04

(57) ABSTRACT

A lens device includes a control module having a control circuit board and a photosensitive element, a light emitting module having a light source circuit board and a light emitting element. The lens module is configured to be corresponsive to the photosensitive element and includes a lens barrel and plural lenses; the focusing lens is installed at a front end of the lens module; the reflector is installed between the lens module and the focusing lens; the hood is connected to the lens barrel and has a shading section extending outwardly from a front edge of the lens barrel for blocking a light reflected by the reflector and prevent the light from entering into the lens barrel directly, so as to avoid poor image quality caused by the light with a poor angle that enters into the lens barrel.

### BACKGROUND OF THE INVENTION

### 1. Technical Field

The technical field relates to a lens module, and more particularly to a lens device with a shading structure.

### 2. Description of Related Art

In general, if a strong light with a poor angle enters into a lens during the use of a camera, the captured image will be of poor quality, or light spots will show up and the overall captured image will be hazy in a worst case scenario. Therefore, a hood is added and installed at the front of a conventional camera to reduce the loss of image quality caused by stray light during shooting and improve the color and saturation of the captured image.

In addition, a commercially available skin testing equipment uses a lens module to take a close-up shot of a skin to be tested, and then performs analysis based on the captured skin image to learn the physiological condition of the skin to be tested. In FIG. 1, a lens device 1a of the conventional skin testing equipment comprises a lens module 10a, a light emitting element 20a, and a photosensitive element 30a, and its operating method is to place a focusing lens 40a near the skin to be tested. A light 21a emitted from the light emitting element 20a passes through the focusing lens 40a and the skin to be tested and then is reflected, and finally the light reflected from the skin to be tested enters into the focusing lens 40a, and passes into the photosensitive element 30a through the lens module 40a to obtain a skin image.

With reference to FIG. 2 for a skin image obtained by the conventional lens device 1a, a light 22a transmitted by the light emitting element 20a of the lens device 1a from a poor angle is reflected by a reflector 50a and enters into the lens module 10a. Such light with the poor angle is one of the main causes for the poor quality of the captured image.

In view of the aforementioned drawback of the prior art, the discloser of this disclosure based on years of experience in the related industry to conduct extensive research and experiment, and finally provided a feasible solution as disclosed in this disclosure to overcome the drawback of the prior art.

### SUMMARY OF THE INVENTION

Therefore, it is a primary object of this disclosure to provide a lens device with a shading structure, and its hood structure includes a shading section extending outwardly from a front edge of a lens barrel for blocking a light reflected from a reflector and preventing the light from entering into a lens barrel directly, so as to avoid the light with a poor angle from entering into the lens barrel to affect the quality of image

In an embodiment of this disclosure, the lens device with a shading structure comprises a control module, a light emitting module, lens module, a focusing lens, a reflector and a hood. The control module comprises a control circuit board and a photosensitive element installed on the control circuit board; the light emitting module comprises a light source circuit board and a light emitting element installed on the light source circuit board, and the light source circuit board is electrically coupled to the control circuit board; the lens module is configured to be corresponsive to the photosensitive element and installed on a side of the control circuit board, and the lens module comprises a lens barrel and a plurality of lenses installed in the lens barrel; the focusing lens is installed at a front end of the lens module; the reflector is installed between the lens module and the focusing lens and capable of reflecting a light emitted by the light emitting element and passing the light out of the focusing lens; the hood is coupled to lens barrel and has a shading section extending outwardly from a front edge of the lens bane and provided for blocking the light reflected by the reflector and prevent the light from entering into the lens barrel directly.

Compared with the prior art, the lens device of this disclosure has a hood structure wherein the shading section extends outwardly from the front edge of the lens barrel to block the light reflected by the reflector and prevent the light from entering into the lens barrel directly. In other words, such structure ensures that the light entering into the lens barrel is the light entering from the focusing lens instead of the light reflected from the reflector, so as to prevent any stray light (which is a light with a poor angle) from entering into the lens barrel and affecting the imaging result and to improve image quality and practicality of this disclosure.

### BRIEF DESCRIPTION OF THE DRAWINGS

FIG. 1 is a schematic view of a light path of a conventional lens device;

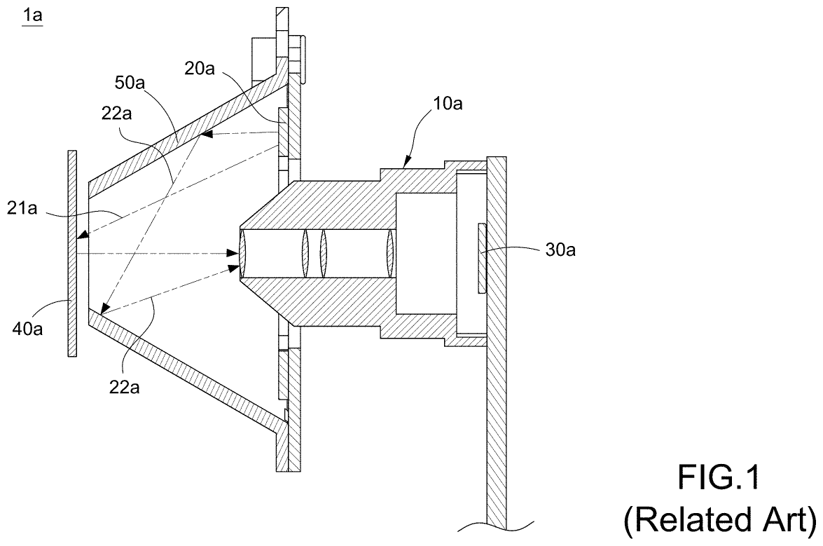

FIG. 2 is a photo of a skin image captured by the conventional lens device;

FIG. 3 is an exploded view of a lens device of this disclosure;

FIG. 4 is a cross-sectional view of a lens device of this disclosure;

FIG. 5 is an exploded view of a lens device applied in a skin testing equipment in accordance with this disclosure;

FIG. 6 is a perspective view of a lens device applied in a skin testing equipment in accordance with this disclosure;

FIG. 7 is a photo of a skin image captured by a lens device of this disclosure; and

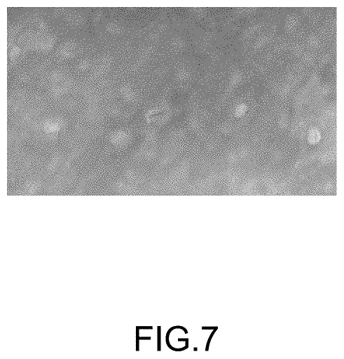

FIG. 8 is a schematic view of a hood of a lens in accordance to another embodiment of this disclosure.

### DESCRIPTION OF THE PREFERRED EMBODIMENTS

The technical contents of this disclosure will become apparent with the detailed description of preferred embodiments accompanied with the illustration of related drawings as follows. It is intended that the embodiments and drawings disclosed herein are to be considered illustrative rather than restrictive.

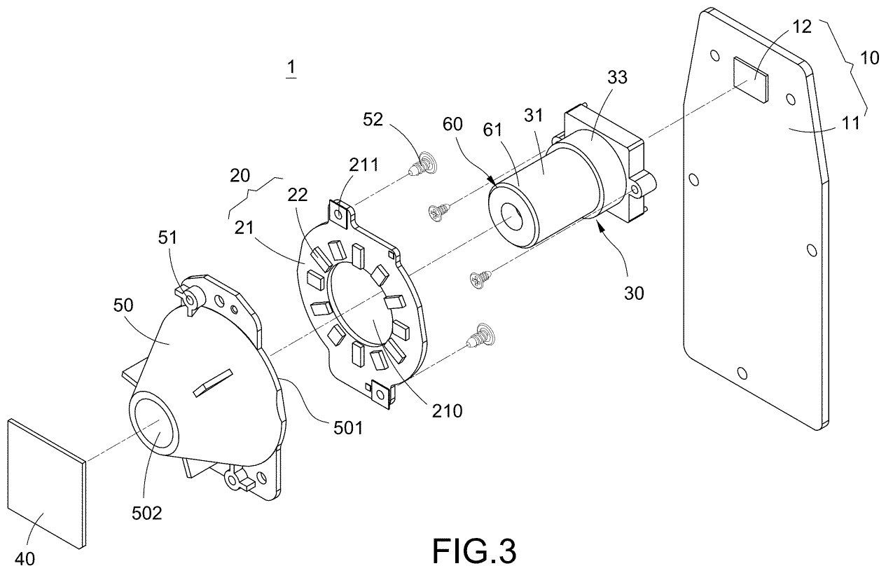

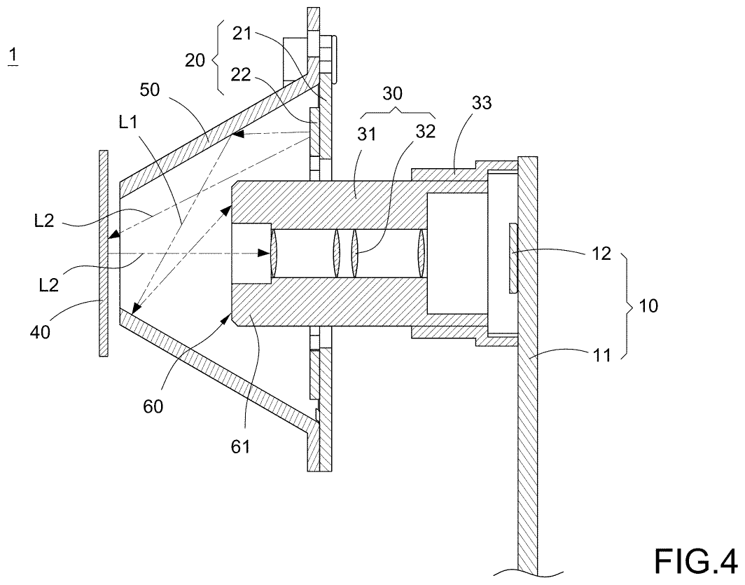

With reference to FIGS. 3 and 4 for an exploded view and cross-sectional view of the lens device with a shading structure in accordance with this disclosure respectively, the lens device with a shading structure 1 comprises a control module 10, a light emitting module 20, a lens module 30, a focusing lens 40, a reflector 50 and a hood 60. The control module 10 is provided for controlling the operation of the light emitting module 20 and receiving the light transmitted from the lens module 30 for computation. Some of the light emitted by the light emitting module 20 will pass out of the focusing lens 40 directly, and some of the light will be reflected by the reflector 50 and then passed out of the focusing lens 40. After the light passing out of the focusing lens 40 and through the object to be tested is reflected, the reflected light passes into the focusing lens 40, and then enters into the lens module 30, and finally is transmitted into the control module 10 for computation, so as to obtain the image of the object to be tested. The detailed structure of the lens device 1 will be described below.

The control module 10 comprises a control circuit board 11 and a photosensitive element 12 installed onto the control circuit board 11.

The light emitting module 20 comprises a light source circuit board 21 and a light emitting element 22 installed onto the light source circuit board 21, and the light source circuit board 21 is electrically coupled to the control circuit board 11. In this embodiment, the light source circuit board 21 has a through hole 210 formed at the center thereof, and the light emitting element 22 comes with a plural quantity, and these light emitting elements 22 are disposed around the periphery of the through hole 210.

The lens module 30 is configured to be corresponsive to the photosensitive element 12 and installed on a side of the control circuit board 11. The lens module 30 comprises a lens barrel 31 and a plurality of lenses 32 installed in the lens barrel 31 (as shown in FIG. 4). In this embodiment, the lens barrel 31 is passed and installed into the through hole 210 of the light source circuit board 21, and the lens module 30 further comprises a lens mount 33. The lens mount 33 is combined with the lens barrel 31 and fixed onto the control circuit board 11.

In addition, the focusing lens 40 is installed at a front end of the lens module 30, and the reflector 50 is installed between the lens module 30 and the focusing lens 40. The reflector 50 is provided for reflecting the light emitted by the light emitting element 22 and passing the light out of the focusing lens 40.

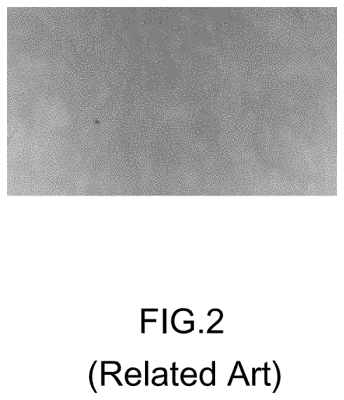

In this embodiment, the hood 60 and the lens barrel 31 are integrally formed. In addition, the hood 60 is coupled to the lens barrel 31 and has a shading section 61 extending outwardly from a front edge of the lens barrel 31. In FIG. 2, the shading section 61 is provided for blocking the light L1 reflected from the reflector 50 and preventing the light L1 from entering into the lens barrel 31 directly. In other words, the installation of the hood 60 ensures that a light such as the light L2 enters into the lens barrel 31 from the focusing lens 40 and preventing any stray light (which is a light with a poor angle) from entering into the lens barrel 31 to affect image quality.

It is noteworthy that the extending length of the shading section 61 can be adjusted according to actual requirements to prevent the light having a poor angle from entering into the lens barrel 31.

Specifically, the light source circuit board 21 has a plurality of screw holes 211, and the reflector 50 has a plurality of coupling holes 51 configured to be corresponsive to the screw holes 211 respectively. The reflector 50 is fixed onto the light source circuit board 21 by passing a plurality of screws 52 into the coupling holes 51 and the screw holes 211.

Specifically, the reflector 50 is substantially in a conical shape, and reflector 50 has a first opening 501 and a second opening 502 opposite to each other, and the first opening 501 abuts against the light source circuit board 21, and the second opening 502 is coupled to the focusing lens 40, and the reflector 60 has an outer contour gradually tapered in a direction from the first opening 501 to the second opening 502.

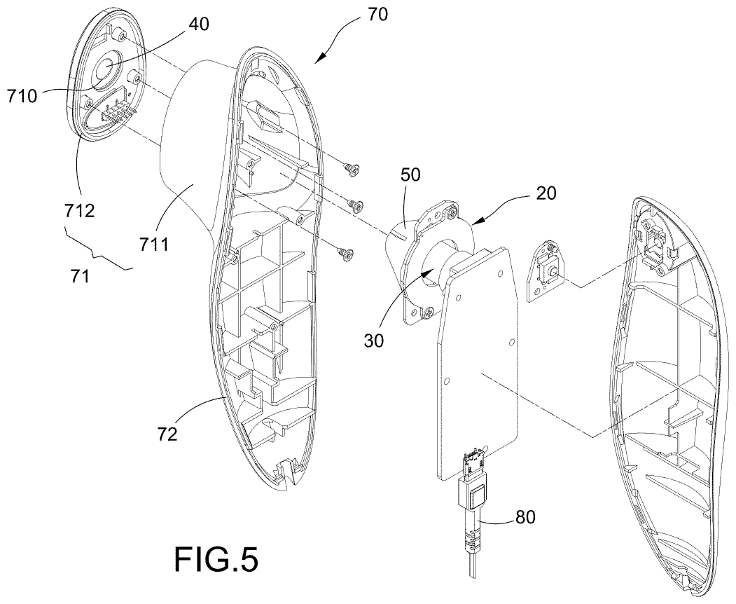

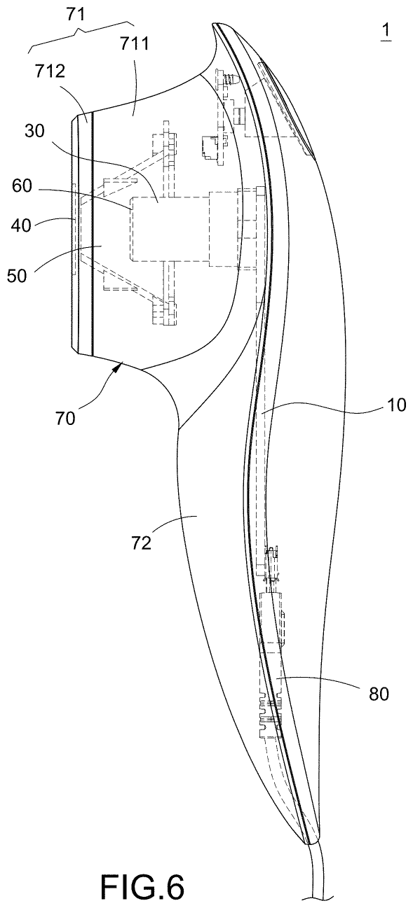

With reference to FIGS. 5 and 6 for an exploded view and a perspective view of a lens device with a shading structure applied to a skin testing equipment in accordance with this disclosure respectively, the lens device is applied to the skin testing equipment as shown in the figures, and the lens device 1 comprises a casing 70 and a cable 80. The control module 10, the light emitting module 20, the lens module 30, the focusing lens 40, the reflector 50 and the hood 60 are installed in the casing 70. In addition, the cable 80 has an end coupled to the control circuit board 11 and the other end passing out of the casing 70 and provided for coupling an external power supply to supply the electric power required for the operation of the lens device 1.

In this embodiment, the casing 70 comprises a detection block 71 and a handle 72 coupled to the detection block 71. The detection block 71 has an opening 710 formed at a front end thereof, and the focusing lens 40 is installed onto the opening 710. Specifically, the detection block 71 comprises a carrying base 711 and a cover 712 for covering the carrying base 711. The carrying base 711 is coupled to the handle 72, and the cover 712 has the opening 710 and combines with the focusing lens 40.

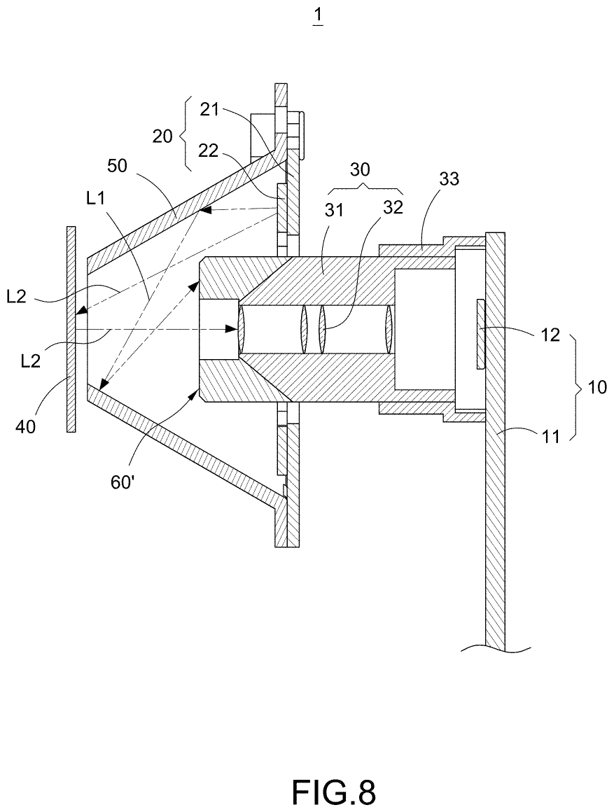

With reference to FIG. 8 for a lens device of another embodiment of this disclosure, the hood 60′ and the lens barrel 31 are two separate components. The hood 60′ is coupled to the lens barrel 31 and extends to the outside from a front edge of the lens barrel 31. Therefore, the hood 60′ can block any light reflected from the reflector 50 and prevent the light from entering into the lens barrel 31 directly, so as to improve the image quality.

It is noteworthy that the length of the hood 60′ extending from the front edge of the lens barrel 31 to the outside can be adjusted according to actual requirements in order to prevent any unnecessary light from entering into the lens barrel 31.

While this disclosure has been described by means of specific embodiments, numerous modifications and variations could be made thereto by those skilled in the art without departing from the scope and spirit of this disclosure set forth in the claims.

What is claimed is:

1. A lens device with a shading structure, comprising:
a control module, comprising a control circuit board and a photosensitive element installed at the control circuit board;
a light emitting module, comprising a light source circuit board and a light emitting element installed at the light source circuit board, and the light source circuit board being electrically coupled to the control circuit board;
a lens module, configured to be corresponsive to the photosensitive element and installed on a side of the control circuit board, and the lens module comprising a lens barrel and a plurality of lenses installed in the lens barrel;
a focusing lens, installed at a front end of the lens modules;
a reflector, installed between the lens module and the focusing lens, and capable of reflecting the light emitted by the light emitting element and passing the light out from the focusing lens; and
a hood, coupled to the lens barrel, and having a shading section outwardly extending from a front edge of the lens barrel, for blocking and preventing the light reflected from the reflector from entering into the lens barrel directly.

2. The lens device with a shading structure as claimed in claim 1, further comprising a casing and a cable, and the control module, the light emitting module, the lens module, the focusing lens, the reflector and the hood being installed in the casing, and the cable having an end coupled to the control circuit board and the other end passing out of the casing.

3. The lens device with a shading structure as claimed in claim 2, wherein the casing comprises a detection block and a handle coupled to the detection block, and an opening is formed at a front end of the detection block and provided for mounting the focusing lens thereon.

4. The lens device with a shading structure as claimed in claim 3, wherein the detection block comprises a carrying base, and a cover covering the carrying base, and the carrying base is coupled to the handle, and the cover has the opening and combines with the focusing lens.

5. The lens device with a shading structure as claimed in claim 1, wherein the light source circuit board has a through hole formed at the center thereof, and the lens barrel is passed and installed into the through hole.

6. The lens device with a shading structure as claimed in claim 1, wherein the light emitting element comes with a plural quantity, and the light emitting elements are disposed around the periphery of the through hole.

7. The lens device with a shading structure as claimed in claim 1, wherein the light source circuit board has a plurality of screw holes, and the reflector has a plurality of coupling holes formed at positions corresponding to the screw holes respectively, and the reflector is fixed onto the light source circuit board by passing a plurality of screws through the coupling holes and the screw holes.

8. The lens device with a shading structure as claimed in claim 7, wherein the reflector has a first opening and a second opening opposite to each other, and the first opening abuts against the light source circuit board, and the second opening connects the focusing lens, and the reflector has an outer contour gradually tapered in a direction from the first opening to the second opening.

9. The lens device with a shading structure as claimed in claim 8, wherein the reflector is substantially in a conical shape.

10. The lens device with a shading structure as claimed in claim 1, wherein the lens module further comprises a lens mount, and the lens mount is combined with the lens barrel and fixed onto the control circuit board.

11. The lens device with a shading structure as claimed in claim 1, wherein the hood and the lens barrel are integrally formed.

12. The lens device with a shading structure as claimed in claim 1, wherein the hood and the lens barrel are two separate components.
